(19) United States
(12) Patent Application Publication
(10) Pub. No.: US 2026/0060516 A1
(43) Pub. Date: Mar. 5, 2026

(54) CapsuleProbe - Application and Method of Capsule Endoscopy in Cervical Examination

(71) Applicants: EMERALD H.F. WANG, CARMEL, IN (US); CHI WANG, CARMEL, IN (US); Katherine Y WANG, CARMEL, IN (US)

(72) Inventors: EMERALD H.F. WANG, CARMEL, IN (US); CHI WANG, CARMEL, IN (US); Katherine Y WANG, CARMEL, IN (US)

(21) Appl. No.: 18/822,086

(22) Filed: Aug. 31, 2024

(51) Int. Cl. A61B 1/04; A61B 1/00; A61B 1/05; A61B 1/06; A61B 1/303

(52) U.S. Cl. CPC A61B 1/041; A61B 1/00016; A61B 1/00032; A61B 1/00071; A61B 1/00147; A61B 1/05; A61B 1/0661; A61B 1/303

(57) ABSTRACT

The invention relates to an apparatus and a method, termed the “CapsuleProbe”, adapted from the capsule endoscopy technology for cervical and/or vaginal examinations. The CapsuleProbe features a capsule endoscope enclosed in a cylindrical housing designed for easy insertion into the vaginal canal. This device provides a minimally invasive and user-friendly option for patients as well as enhanced visualization of the cervix for healthcare providers. With its substantially minimized dimensions comparing to existing medical devices for cervical and/or vaginal examinations, this device improves feasibility, accessibility, and patient comfort significantly, particularly for postmenopausal women and those suffering from vaginal atrophy, by reducing or eliminating procedure-related pain and/or bleeding from the tearing of vaginal walls caused by existing invasive medical devices used for cervical and/or vaginal examinations.

### FIELD OF THE INVENTION

The present invention relates to the field of medical diagnostic devices, specifically to a novel application and method of utilizing standalone pill-size capsule endoscopy for cervical examination. This invention pertains to a specially designed “CapsuleProbe”, intended for use in gynecological diagnostics initially with broader applications.

### BACKGROUND OF THE INVENTION AND PRIOR ART

Standalone capsule endoscopy, using a PillCam—a pill-size ingestible camara, has revolutionized the examination of the gastrointestinal tract, allowing for minimally invasive diagnostics. However, capsule endoscopy technologies have not been adapted for gynecological applications, particularly for cervical examinations. Traditional methods of cervical examination, such as Pap smears and colposcopy, though effective, can be painful and even physically damaging for postmenopausal women and patients with vaginal atrophy. These challenges can discourage or even prevent affected women from undergoing regular or timely cervical exam, therefore increase their risks of missing early detection of cervical cancer.

### Prevalence of Vaginal Atrophy and the Need for Improved Examination Methods

Vaginal atrophy is a common condition among postmenopausal women, affecting approximately 50% of this population. This condition, characterized by thinning, drying, and inflammation of the vaginal walls, is associated with decreased estrogen levels during menopause. Vaginal atrophy can lead to symptoms such as discomfort, pain, and increased vulnerability to physical tearing, particularly during vaginal examinations.

The population of women aged 50 and older is significant in the US including Boomers and a large portion of Gen-X, with global trends indicating a significant rise in the number of postmenopausal women in the coming decades. As a result, there is a growing need for medical devices and examination methods that are tailored to the unique physiological changes that occur in this demographic.

### Challenges With Traditional Vaginal Examinations

The Vaginal Speculum 100A (FIG. 5), a widely used instrument for gynecological examinations, presents several challenges, especially for postmenopausal women. The speculum is typically large, hard, and sharp-edged, which can cause significant discomfort, pain, and even physical tearing of the vaginal walls during insertion and examination. These issues are exacerbated in women with vaginal atrophy, where the vaginal tissue is more delicate and prone to injury.

Another less common but more expensive procedure is transvaginal ultrasound. Again, for postmenopausal women coping with vaginal atrophy, this procedure can be painful and physically damaging since the size of the transducer probe 100B (FIG. 6) is as large as a regular vaginal speculum. It can cause tearing of vaginal walls and sometimes fail to be pushed into the vaginal canal due to its large size for petite women.

Given these challenges, there is an urgent need for alternative examination devices and methods that are less invasive and more comfortable for patients. The present invention addresses this need by introducing a novel device using a capsule endoscope enclosed in a tampon-sized housing. This device allows for enhanced visualization and examination of the cervix in a minimally invasive manner with much improved ease of use and patient comfort, particularly for women suffering from vaginal atrophy.

### SUMMARY OF THE INVENTION

The present invention relates to an apparatus and method for cervical examination, comprising a medical imaging device referred to herein as a “CapsuleProbe.” The CapsuleProbe includes a pill-size capsule endoscope secured within an portable, disposable, cylindrical housing with the size corresponding to a regular tampon. Comparing to existing large invasive devices used in cervical examination, the tampon-sized housing is configured for easy insertion into the vaginal canal to minimize physical trauma to the vaginal wall, and to position the capsule endoscope next to the cervix for imaging.

By adapting pill-size capsule endoscopy for gynecological purposes, and with a substantially minimized dimensional size compared to existing arts, the present invention is configured to provide a much more comfortable and drastically less invasive alternative to conventional cervical examination techniques, for improving patient compliance with early cervical health screening and cancer prevention. The structure and functionality described herein are sufficient to enable a person skilled in the art to make and use the invention without undue experimentation.

The use of the present invention is applicable for additional bodily cavity applications with throat or anal canal examinations.

The invention includes the following key components:
1. Capsule endoscope: An existing capsule endoscope.
2. Elongated Cylindrical Housing: A cylindrical housing unit with the size corresponding to a regular tampon, designed to hold a pill-size capsule endoscope at the distal end of the housing unit enclosed by a transparent dome cover, to be inserted into the vaginal canal and placed next to the cervix. This housing is made from a biocompatible material and is shaped round and smooth to allow for easy and comfortable insertion into the vaginal canal. The housing is made of disposable tampon materials of biocompatible plastics or composite fibers, or of medical-grade plastic, silicone, or alloys designed for repeated use after sterilization for economical purposes. The transparent dome cover is made of clear medical-grade plastics.

### OBJECTS OF THE INVENTION

1. Patient Comfort: To provide a n alternative device and a method for cervical examination that is much less invasive and more comfortable for patients compared to traditional devices and methods, particularly for women with vaginal atrophy.
2. Enhanced Visualization: To offer a device that provides enhanced visualization of the cervix, leading to improved diagnostic capabilities.
3. Ease of Use: To design a portable miniature device that can be easily used by patients themselves or healthcare providers, applicable for both home and medical office settings.
4. Injury Prevention: To create a device with significantly minimized dimensions that reduces the need for invasive procedures and associated physical trauma—tearing of the vaginal wall.

### BRIEF DESCRIPTION OF THE DRAWINGS

FIG. 1 depicts the general shape, look, and the camera head of a capsule endoscope.

FIG. 2 illustrates perspective views of the device and its components.

FIG. 3 shows a side section view of the device in use, placed inside of vaginal canal and next to the cervix.

FIG. 4 is a flowchart for the method of use.

FIG. 5 depicts a Vaginal Speculum referenced in the Background/Prior Art section.

FIG. 6 illustrates the size of a Transvaginal Ultrasound Transducer Probe as large as a Vaginal Speculum, referenced in the Background/Prior Art section.

### DETAILED DESCRIPTION OF THE INVENTION

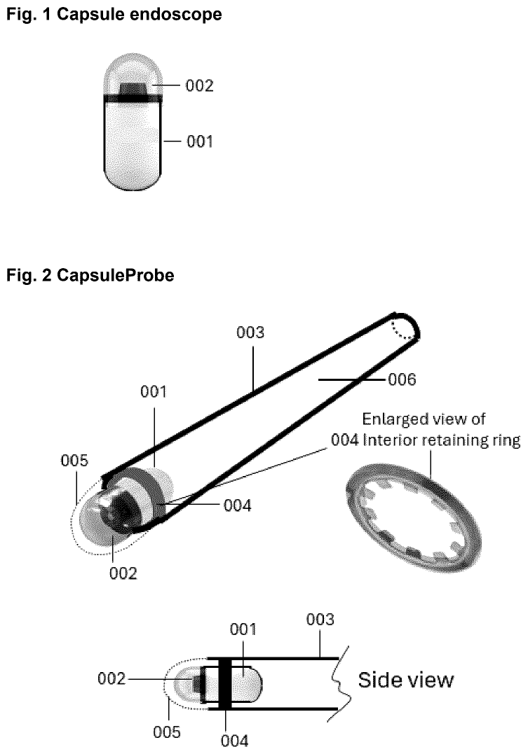

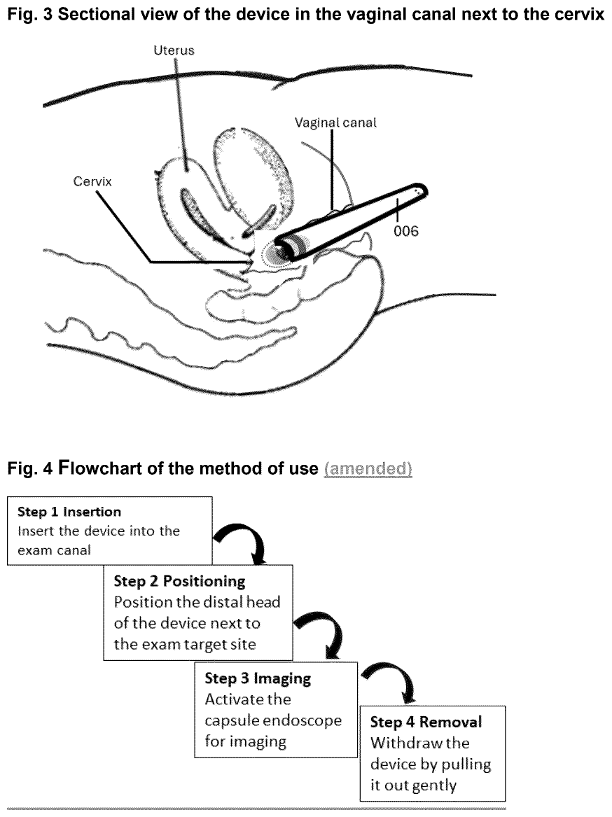

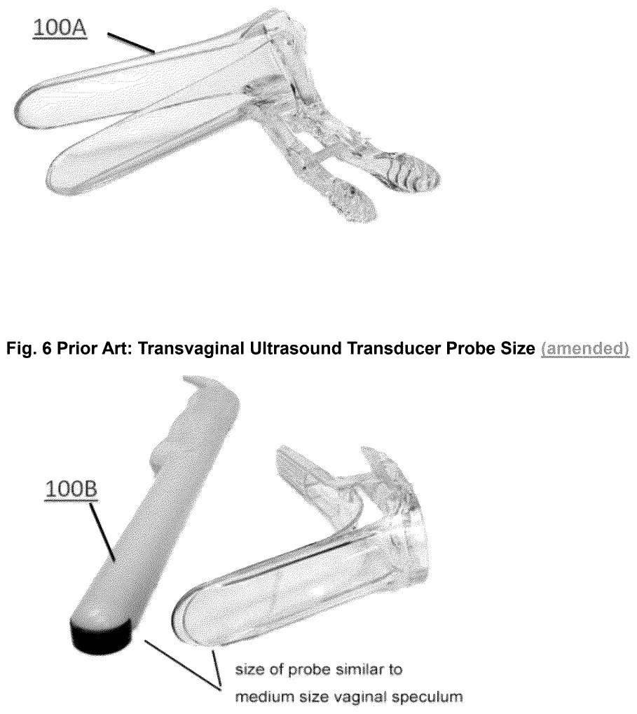

Capsule Endoscope: The device 006 (FIGS. 2, 3) utilizes existing technology of a capsule endoscope 001 (FIGS. 1, 2) as its imaging capturing and transferring component. The camera head 002 of the capsule endoscope 001 is equipped with a high-resolution camera and light source, allowing for detailed imaging of the cervix in this application. The capsule itself is powered by a small internal battery and can take and transmit real-time video footage or pictures wirelessly to an external recorder or monitor.

Housing: The elongated slim cylindrical housing 003 forms the body of the device 006. It holds and places capsule endoscope 001 through the vaginal canal to reach the cervix. The housing 003 is configured with the size corresponding to a regular tampon and is made of biocompatible disposable tampon housing materials, or reusable medical-grade plastics, medical-grade silicone or alloys. The exterior of the housing is smooth and rounded, ensuring easy and comfortable insertion and removal.

Retaining ring: The housing 003 features an interior retaining ring 004 made from strong and elastic materials of silicone, rubber, foam, or flexible metals. It is fixed to the interior wall of the housing 003 by strong medical-grade glue, snap-fit with an internal groove, or male-female threads. The retaining ring 004 securely holds the capsule endoscope 001 with its camera head 002 facing outward at the distal end of the housing 003.

Transparent dome cover: The camera head 002 of the capsule endoscope 001 is enclosed under a transparent dome cover 005 made from clear medical-grade plastics. The dome cover 005 is attached to the housing 003 body by strong medical-grade glue, male-female threads, or snap-fit. The dome cover 005 prevents the capsule endoscope 001 from getting trapped inside of the vaginal canal in the event of it becoming loose from the retaining ring or glues.

Easy usage: The device 006 is configured for comfortable use as easy as placing a tampon in the vaginal canal. Once inserted into the vaginal canal, the device 006 positions the enclosed capsule endoscope next to the cervix (FIG. 3) for imaging. The patient or healthcare provider conducts the exam and retrieves the device 006 easily by pulling it out gently, either in a home or medical office setting, as the captured images are transmitted to a remote monitor.

### 3. Method of Use FIG. 4

The method of use for the device is as follows:
1. Insertion: The patient or healthcare provider inserts the device into the examination site.
2. Positioning: Gently push the device to position its distal end next to the target site.
3. Imaging: The device begins capturing and transmitting images for diagnostic purposes.
4. Removal: After the imaging is completed, the patient or healthcare provider withdraws the device by pulling it out gently.

1. A medical device for cervical examination comprising:
a capsule endoscope including a camera, a light source, an internal power source, and wireless transmission capability;
a smooth cylindrical housing unit made from biocompatible material and with the size corresponding to a regular tampon;
an internal retaining ring positioned within the cylindrical housing, configured to securely hold the capsule endoscope with the camera oriented outwardly towards a transparent dome cover at the distal end of the housing; and
the transparent dome cover attached to the distal end of the housing, configured to safeguard the capsule endoscope from detachment and to minimize insertion trauma.

2. The medical device of claim 1, wherein the cylindrical housing is composed of the same disposable biocompatible plastics or composite fibers used for conventional tampons, or medical-grade plastics, silicone, or alloys for sterilization and reuse.

3. The medical device of claim 1, wherein the internal retaining ring is made from strong and elastic materials of silicone, rubber, foam, or flexible metals.

4. The medical device of claim 1, wherein the internal retaining ring is fixed to the interior wall of the housing by strong medical-grade glue, snap-fit with an internal groove, or male-female threads.

5. The medical device of claim 1, wherein the transparent dome cover is securely fastened to the housing by strong medical-grade glue, male-female threads, or snap-fit.

6. A method of cervical examination using the medical device of claim 1, comprising:
inserting the medical device gently into the vaginal canal by the patient or her healthcare provider;
positioning the cylindrical housing so the transparent dome cover is next to the cervix;
activating the capsule endoscope to capture images or video footage of the cervix and vaginal walls, wherein said images are wirelessly transmitted to an external receiver or monitor;
removing the medical device from the vaginal canal by the patient or her healthcare provider after image capture is completed.

7. Additional methods of use of the medical device of claim 1 include throat or anal canal examinations.
